(19) United States
(12) Patent Application Publication
(10) Pub. No.: US 2012/0278956 A1
(43) Pub. Date: Nov. 1, 2012

(54) SOIL FREE PLANTING COMPOSITION

(76) Inventor: Gary R. Hartman, Hollister, CA (US)

(21) Appl. No.: 13/456,442

(22) Filed: Apr. 26, 2012

Related U.S. Application Data
(60) Provisional application No. 61/479,013, filed on Apr. 26, 2011.

(51) Int. Cl. C08L 75/08; A01N 43/78; A01H 5/00; A01P 13/00; A01P 1/00; A01P 3/00; A01P 7/00; C08L 75/06; A01N 55/10

(52) U.S. Cl. 800/323; 521/174; 521/172; 514/63; 514/365; 800/298; 504/123

(57) ABSTRACT

Plant growth substrates that lack organic components, such as organic soil, peat or bark material. In certain aspects sponge-like matrix materials are provided that are porous, retains water and can be used to maintain plant growth. Matrix materials, for instance, can comprise an admixture of a hydrophilic polymer, such as a polyurethane, and an amorphous silica. Methods of growing and maintaining plants and plant parts in such materials are also provided.

This application claims the benefit of U.S. Provisional Patent Application No. 61/479,013, filed Apr. 26, 2011, the entirety of which is incorporated herein by reference.

### BACKGROUND OF THE INVENTION

1. Field of the Invention

The present invention relates generally to the fields of horticulture and agriculture.

2. Description of Related Art

In nature, as well as in cultivated fields and greenhouses, plants are maintained in a variety of complex organic soils. In general these soils provide a medium from which the roots of a plant can absorb nutrients and water. Soil from different sources or locations can, however, vary significantly in the nutrient and moisture content resulting in variable plant growth characteristics. Additionally, due to its undefined nature, soil can harbor various undesirable contaminates such a pesticides, bacteria, insects, viruses and fungi. However, the complex nature of soil and varying needs of plants for growth and survival make even the most simple soils refractory to substitution with synthetic substrates.

A number of attempts have been made at producing plant growth media that include various synthetic components. However, even these partially synthetic plant media do not provide the characteristics need for a true substitute for organic soil. For example, plug propagation system such as Fertiss® are still composed of undefined organic components such a peat moss. Growth media formulations, such as those described in U.S. Pat. No. 7,832,145, require organics and a liquid bath as a source of water and nutrients. Likewise, the polymer formulations described in U.S. Pat. No. 6,032,412 require organic additives to support moisture retention and plant growth.

Conversely, truly synthetic media, such agar based systems that have been used for experimental plant cell culture, have little real world applicability because they are unable to maintain moisture levels outside of a controlled laboratory environment. Additionally, these agar based systems require a large number of constituents that result in high production cost. Thus, there existed a need for plant growth substrates that can sustain plant health without the need of undefined organic components.

### SUMMARY OF THE INVENTION

In a first embodiment, the invention provides a sponge-like matrix that is porous, retains water and can be used to maintain plant growth, wherein the matrix is essentially free of organic soil, peat, coir, humus and/or bark material. The matrix, for instance, can comprise an admixture of a hydrophilic polymer and an amorphous silica. For example, one or more amorphous silica components can be mixed with hydrophilic polymer subunits prior to polymerization to provide a sponge-like matrix comprising amorphous silica dispersed through-out the matrix. Additional components can be incorporated into a matrix according to the embodiments (either before, during or after the polymer subunits have been polymerized). A matrix according to the invention is substantially porous thereby maintain substantial water and air content within the matrix. For example, a matrix can comprise an average porosity of between about 10 and 300 pores per inch (ppi).

In certain aspects, a sponge-like matrix according to the embodiments is mechanically resilient and can return to its original shape following mechanical compression (e.g., the matrix can be defined as a memory foam). In still further aspects, a sponge-like matrix is substantially non-friable. For example, a matrix according to the embodiments can, in some aspects, be cut without a significant portion of the matrix crumbling-away.

A variety of hydrophilic polymers are known in the art, which may be used in a matrix according to the invention. In certain aspects, the matrix comprises a polyurethane polymer, such as a polymer of a polyol and a isocyanate (e.g., a diisocyanate). These subunits, once polymerized form a cross-linked web of polar polymer strands that can maintain water content. In certain aspects, the matrix can be defined by the size of the molecules between the cross linking bonds. For example, in certain aspects, the polymer can be defined by the equivalent weight per NCO, such as a polymer comprising an equivalent weight of between about 100 and 1,000 per NCO (e.g., about 300, 400 or 500 to about 700).

As detailed herein, in certain aspects, isocyanates form part of a hydrophilic polymer matrix according to the invention. The isocyanate can be, without limitation, methylene diphenyl diisocyanate (MDI), toluene diisocyanate (TDI), hexamethylene diisocyanate (HDI) and/or isophorone diisocyanate (IPDI). For example, a MDI polymer may be formed from 2,2′-MDI, 2,4′-MDI, 4,4′-MDI or a mixture thereof. Monomeric or polymeric MDI can, for example, be reacted with polyols to form MDI-based polyurethanes. Likewise, in certain aspects, the polymer is a TDI-based polymer, such a polymer formed by 2,4-TDI, 2,6-TDI or a mixture thereof. For instance, the polymer may be formed from a mixture of a 2,4-TDI and 2,6-TDI at a ratio of about 80:20, 70:30, 60:40 or 65:35.

In further aspects, a hydrophilic polymer is formed from polyol component molecules, such as polymeric polyols (e.g., a polyether or polyester). Thus, in certain aspects a hydrophilic matrix comprises a polyether and/or polyester linkages. The polyol component can, in certain aspects, be characterized by a molecular weight (MW) of between about 250 and 10,000 (e.g., about 1,000, 1,500, 2,000, 2,500, 3,000, 3,500, 4,000, 4,500, 5,000, 6,000, 7,000, 8,000 or 9,000).

In certain embodiments, a sponge-like matrix comprises one or more amorphous silica component(s). In some aspects, the amorphous silica is dispersed homogenously throughout the polymer matrix. The amorphous silica component can, for example, be vermiculite, biotite, phlogopite, mica, perlite, hydrated obsidian, diatomaceous earth or a mixture thereof. In certain aspects, the amorphous silica is a hydrated silica, such as hydrated vermiculite or perlite. In still further aspects, expanded silicas may be used, such as expanded vermiculite and/or perlite.

A matrix according the instant invention can be provided in virtually any shape, by for example, polymerizing the matrix in a mold of a desirable shape or by cutting or milling the matrix after polymerization. For instance, the matrix can be provided as a cup, pot, slab or plug shape. In certain aspects, a matrix is provided as plug comprising a centrally disposed cavity (e.g., a hole, which extends through all or a portion of the plug). In further aspects, a matrix is provided as a plug comprising a slice that bisects all or a portion of the plug. A matrix plug can be formed into virtually any size, such as, for example, a plug having a diameter of between about 0.25 to 5 inches and a height of between about 0.5 to 12 inches. Example plug shapes and dimensions are provided, for instance, in U.S. Pat. No. 6,901,699, incorporated herein by reference. In still further aspects, a plurality of individual pieces of matrix (e.g., plugs of matrix) are arranged in a strip or tray and be comprised in a support material, such as a plastic.

As detailed herein a sponge-like matrix according to the invention may comprise additional components. For example, in certain cases, the matrix can comprise components that support plant cell survival and/or growth (e.g., fertilizers or minerals). In still further aspects, components such as surfactants can be added that facilitate or alter matrix polymerization. Examples of additional components that can be comprised in a matrix include, without limitation, a nitrogen source (e.g., an ammonium or nitrate salt), a phosphorus source, a pH adjusting agent (e.g., lime to reduce pH), a natural or synthetic fiber, a water holding/releasing agent, a surfactant, an antioxidant, a pesticide, an herbicide, an antibiotic, a plant hormone (e.g., a rooting hormone), a soil conditioning agent (e.g., clay, diatomaceous earth, crushed stone, a hydrogel, or gypsum) or an antifungal agent.

In yet a further embodiment, a matrix according to the invention comprises plant or plant part. For example, a matrix can comprise a seed, seeding, a cutting or a callus culture from a plant. A plant or plant part embedded or associated with a matrix may be a moncot or a dicot. In certain aspects, the plant is a plant that can be vegetatively propagated. In some aspects, a matrix comprises a plant or plant part of an ornamental plant (e.g., a poinsettia, impatiens or geranium), a landscaping plant, an herb, a garden vegetable or a fruit or nut tree. In further aspects, a single plant or living portion thereof is provided in each piece (e.g., plug) of matrix. Thus, a plurality of plants can be provided, each in a separate plug of matrix, wherein the plurality of matrix plugs and plants are supported on a strip or tray.

In still a further embodiment the invention provides a method for growing a plant comprising positioning a plant in a matrix according to the embodiments and allowing the plant to grow. Thus, a plant is positioned in the matrix such that the matrix can provide water and nutrients to the plant to allow plant growth and/or survival. For example, a plant part can be positioned in a cavity in a matrix, such that the plant is in contact with the matrix (e.g., a portion of a plant or cutting can be embedded in the matrix). In the case of a plug-shaped matrix, for example, the plant part can be positioned in a hole near the center of the plug or the plug can be cut and the plant folded into the cut of the matrix. A matrix comprising a plant part can be maintained in conditions that are favorable for plant growth or survival. For example, a plant can be grown in a lighted environment with appropriate humidity and temperature such as in a hydroponic system, a greenhouse or outdoor field. Thus, in a related embodiment, the invention provides a method for maintain plant health comprising positioning a plant or plant part in a matrix according to the invention such that the plant is provided with water and nutrients by the matrix thereby maintaining plant health.

In certain aspects, a method for growing a plant can be defined as a method for establishing roots, such as by maintaining a plant in a matrix under conditions that favor root tissue formation. For example, in certain aspects, an in vitro plant culture is positioned in a matrix under conditions permissive for root tissue formation. In some cases, plant hormones or growth regulators can be added to the matrix to favor plant growth and/or root tissue formation.

In yet a further embodiment, there is provided a method for manipulating a sponge-like matrix according to the embodiments. In certain aspects, manipulating a sponge-like matrix can comprise moving a matrix from a first container to a second container. For example, manipulating the sponge-like matrix can comprise moving the matrix by an automated process (e.g., by use of a robotic machine comprising a gripper for contacting the matrix). Thus, in certain cases, a method for transplanting a plant is provided comprising obtaining a first container comprising a plant positioned in a sponge-like matrix and transplanting the plant and matrix to second container. The transplanting can be accomplished, for example, by using a robotic transplanting machine. A robotic transplanting machine can, for instance, be a computer controlled machine. Example robotic machines for transplanting plants are described in U.S. Patent Publn. Nos. 20040020110 and 20120005955 and in Dutch Patent Appln. No. NL-2004951, filed Jun. 23, 2010, each of which is incorporated herein by reference.

In still a further embodiment there is provided a method for making a sponge-like matrix according to the invention comprising obtaining a slurry of amorphous silica and hydrophilic polymer subunits, wherein the slurry is essentially free of organic soil, peat, coir, humus and/or bark material; allowing the hydrophilic polymer subunits to polymerize and thereby form a sponge-like matrix that is porous, retains water and can be used to maintain plant growth. For example, a slurry can be mixed to provide an essentially homogenous distribution of the amorphous silica throughout and placed into a mold as polymerization occurs. In certain aspects such mixing is performed at reduced pressure. Mechanical mixers that may be used are, for example described in U.S. Patent Publn. 20080035217, incorporated herein by reference.

In certain embodiments, methods according to the invention involve obtaining a slurry of amorphous silica and a hydrophilic polymer subunits. A slurry for use herein comprises particles of amorphous silica dispersed in an aqueous solution. For instance, in certain aspects the slurry comprises about 10%-70%, by volume of an amorphous silica (e.g., about 20%, 25%, 30%, 35%, or 40% to about 60% by volume). Thus, in some aspects, the slurry comprises about 30%, 40%, 50%, or 60% by volume of the amorphous silica component. In certain cases, the amorphous silica in a slurry is comprised of two or more type of amorphous silica. For example, a slurry can comprise a mixture of vermiculite and perlite. Such mixtures of two or more types of amorphous silica may be formulated at various ratios such as, for example, 1:1, 1:2, 1:3, 1:4, 1:5, 1:10 or 1:20.

In further aspects, a slurry for use according to the invention comprises hydrophilic polymer subunits. Subunits for any of the hydrophilic polymers described herein or known in the art may be used in such a slurry. For example, in the case of a polyurethane polymer, a slurry may comprise an isocyanate (e.g., a diisocyanate) and a polyol, such as a polymeric polyol. For example, a slurry can comprise a solution comprising about 2%, 3%, 4%, 5%, 6%, 7%, 8%, 9%, 10%, 11%, 12%, 13%, 14%, 15% to about 20% weight/weight of polymer subunits.

In still further embodiments a slurry according to the invention comprises additional components either dispersed or dissolved into the slurry. For example, the slurry can comprise a nitrogen source, a phosphorous source, a surfactant, a pesticide, an herbicide, an antibiotic or an antifungal agent. In certain cases, the slurry may be defined as essentially free from organic material.

As detailed above, in certain aspects a slurry according to the invention is allowed to polymerize. For example, in certain aspects, the slurry is placed in a mold before the polymerization is complete. In this case, the mold shapes the sponge-like polymer into a desirable shape (e.g., in the shape of a cup, pot, slab or plug). In further aspects, a matrix material is processed into its final shape after polymerization is complete. For example, the matrix can be cut, shaved or compressed into the desired shape.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawing forms part of the present specification and is included to further demonstrate certain aspects of the present invention. The invention may be better understood by reference the drawing in combination with the detailed description of specific embodiments presented herein.

FIG. 1: A tray with plugs of example sponge-like matrix material for plant growth.

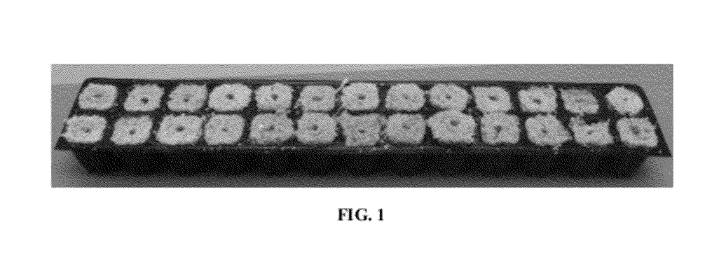

FIG. 2: Graph shows results from water retention studies with a sponge-like matrix material of the embodiments termed “Ecke”. Bar graphs indicate the volume percent of solids, water or air (y axis) when the material is at water container capacity (CC), dry or under 2, 10 or 50 centibars of pressure (x axis), as indicated.

FIG. 3: Graph shows results from water retention studies with a plant media material termed “Oasis”. Bar graphs indicate the volume percent of solids, water or air (y axis) when the material is at water container capacity (CC), dry or under 2, 10 or 50 centibars of pressure (x axis), as indicated.

FIG. 4: Graph shows results from water retention studies with a plant media material termed “Rockwool”. Bar graphs indicate the volume percent of solids, water or air (y axis) when the material is at water container capacity (CC), dry or under 2, 10 or 50 centibars of pressure (x axis), as indicated.

### DESCRIPTION OF ILLUSTRATIVE EMBODIMENTS

Plant growth substrates that are currently available include a range of undefined organic components such as soil, humus and peat. However, substrates that include such undefined organic components are undesirable because they may include contaminating organisms or chemicals. Additionally, the import of organic, e.g., soil-based compositions, into various jurisdictions is restricted, which reduces the usefulness of these compositions for distribution of plant material. On the other hand, fully synthetic materials have not been previously formulated that can provide an adequate substrate to maintain and grow plants and cuttings. In particular, without organic components moisture levels required to sustain plant health cannot be maintained.

The materials described herein provide for first time a plant growth substrate that allows adequate moisture and nutrient delivery to plants without the need for soil, peat, coir, or humus. Matrix materials can comprise a hydrophilic polymer, such as a polyurethane with incorporated amorphous silica components. Because the hydrophilic sponge-like matrix allows incorporation of a significant portion of water and nutrients directly into the matrix additional nutrient solutions do not need to be added to support plant growth.

The attributes of a matrix according to the invention affords a number of unique advantages to the resulting plant growth substrates. For example, the hydrophilic matrix provides both excellent water retention and adequate air porosity to maintain plant health. Thus, the plant material is supplied with adequate water to prevent desiccation, while air throughout prevents rotting of exposed plant tissues. The foam substructure with-in the substrates allows water and nutrients to move through the substrate for sufficient and sustained delivery to a plant that is positioned in the substrate. Thus, the matrix does not need to be maintained in a bath of water and nutrients. For example, even small portions of this new matrix (e.g., plugs less than two inched in a diameter) can maintain moisture for extended periods of time. Thus, even matrix that is formed into shapes that have a high surface area to volume ratio (such as small plugs) are able to maintain sufficient moisture to maintain the health of embedded plants

The formable nature of the matrix materials described here allows virtually any shape or size of substrate to be made. Specifically, the materials can be mixed (along with other components for matrix incorporation) into a slurry and cast into a mold for formation. Thus, matrix substrates can be produced en mass while maintaining virtually identical formulation for all of the substrates produced. Such mass-produced matrix can be used, for instance, in packing of plants and plant parts at any required density with homogenous distribution of water and nutrients to each of the individual plants.

The mechanical properties of the sponge-like matrix described herein also offer significant advantages. These matrix substrates are substantially non-friable and thus can be cut into any required shape without a large portion of the matrix crumbling-away. For example, a plug formed from the matrix can be bisected with a cut and then a plant placed in the cut portion of the matrix such that it is in direct contact with the matrix (and the thus the moisture and nutrients provided by the matrix). The substantially non-friable property of the matrix thereby allows for easy placement and removal of both rooted and un-rooted plants without significant damage to the plant tissue.

The matrix according to the embodiments is also mechanically resilient and retains memory of its original shape after mechanical compression. This property is crucial to high-throughput processing and manipulation the matrix. For example, the matrix (and any embedded plant) can be manipulated with a robotic machine that uses gripper elements. In this case, the grippers can depress the matrix in order to manipulate it (e.g., move the matrix to a new location), however, after the gripper releases the matrix will return to its original shape. This allows any embedded plant to be moved by an automated robot with-out damage to the plant that could occur without such a resilient matrix. Likewise, the ability of the matrix to be mechanically depressed allows robotic arms to effectively grip and position portions for the matrix with a high degree of accuracy and reproducibility.

Additional aspects to the plant growth substrates are detailed below.

### I. Hydrophilic Polymer Matrix

A wide variety of hydrophilic polymers are known and can be used to form the sponge-like matrix according to the instant invention. Polymers can be formed from prepolymer subunits that are formulated de novo, however, a variety of prepolymer mixtures are commercially available and can be used according to the invention.

For example, polyurethane prepolymers comprising a polyol and an isocyanate (e.g., diisocyanates) may be used in a polymer matrix. Such prepolymers can be purchased from a variety of suppliers and can be mixed with water for polymer formation. The resulting polymers form foams and hydrogels that can comprise many times their dry weight in water (e.g., up to 90% water). Depending on the mixing procedure polymer formation typically occurs in 5-10 minutes. Components incorporated or dispersed in the prepolymer mixture, may also, therefore by incorporated into the polymer matrix. In general, prepolymer mixtures are available as liquid resins. Polymers are produced by reacting polyols (e.g., low molecular weight polyols with 3-8 hydroxyl groups) with aromatic or aliphatic diisocyanates. After the reaction, the resins have at least two free isocyanate groups per molecule of polyol used.

Isocyanate Polymers

Isocyanates that may used include MDI, TDI, HDI, IPDI or a mixture thereof. Exemplary isocyanate-capped polyether prepolymers have previously been described for example in U.S. Pat. Nos. 3,903,232, 4,137,200, 4,517,326, 5,650,450 and 5,916,928, each of which are incorporated herein by reference.

Prepolymers are defined, in some aspects, by the average isocyanate functionality, such as a functionality greater than 2. A wide range of prepolymer mixtures can be formulated or are commercially available and can be defined by the physical properties of the polymers that they form. These prepolymer mixes are typically made from varying ratios of a polyalkylene glycol and a polyhydricalcohol containing 3 or 4 hydroxyl groups per molecule with enough TDI (or MDI) to cap all of the hydroxyl groups. The formulations can be defined by the weight of the polymer molecules between NCO branch points (per NCO), the relative NCO content, specific gravity and viscosity. For example, a formulation can comprise an isocyanate-capped polyoxyethylene polyol polyurethane prepolymer derived from TDI a MW of 625 per NCO, an NCO content of 1.60 meq/g, a specific gravity of 1.19 and a viscosity of 18,500-20,000 cps (Brookfield LVF, #4 Spindle, 12 rpm at 25° C.). Another exemplary formulation is a TDI-based formulation comprising an equivalent weight (per NCO) of 425, an NCO content of 2.35 meq/g, a specific gravity of 1.15 and a viscosity (measured as described above) of 10,500.

Still further prepolymer formulation that may be used according to the invention include, but are not limited to: (1) an isocyanate-capped polyoxyethylene polyol polyurethane prepolymer derived from TDI having an NCO content of 0.5-0.9 meq/g. and a viscosity at 25° C. of 10,000 to 12,000 cps, (2) an isocyanate-capped polyoxyethylene polyol polyurethane prepolymer derived from isophorone diisocyanate having an NCO content of 1.8 meq/gram and a viscosity at 25° C. of 12,000 cps; (3) an isocyanate-capped polyoxyethylene polyol polyurethane prepolymer derived from TDI having an NCO content of 1.4 meq/gram and a viscosity at 90° C. of 4,700 cps; (4) an isocyanate-capped polyoxyethylene polyol polyurethane prepolymer derived from methylenediphenyl diisocyanate having an NCO content of 2.55 meq/g, an equivalent weight (per-NCO group) of 392 and a viscosity at 25° C. of 18,000 cps; and (5) an isocyanate-capped polyoxyethylene polyol polyurethane prepolymer derived from methylenediphenyl diisocyanate having an equivalent weight (per-NCO group) of 476, an NCO content of 2.10 meq/g and a viscosity at 25° C. of 20,000 cps.

Yet further TDI prepolymers are available that comprise an NCO-value of 2.5 to 3.0 and are formed from the reaction of toluene diisocyanate and an organic polyether polyol containing at least 40 percent by weight ethylene oxide adducts (see, e.g., U.S. Pat. No. 4,517,326).

Polyols

A polyol, such as polyether polyol, component of a matrix should preferably have a functionality of 2 to 6, an average molecular weight in the range from 250 to 12,000, such as from about 350 to 6000. A polyether polyol component may comprise at least one polyether which contains an amino-group. Such a polyether polyol component may contain aminopolyethers which comprise propyleneoxy or ethyleneoxy groups, and which are started on triethanolamine or ethylenediamine.

Further polyols that may be used according to the invention include, hydrophilic oxyalkylene polyols or diols (such as ethylene glycol, 1,2-propylene glycol, 1,3-propylene glycol, diethylene glycol, 1,4-butane diol, 1,3-butane diol, 1,6-hexane diol, 2-ethyl-1,3-hexane diol and others).

Amorphous Silica

Matrix substrates according the invention comprise one or more amorphous silica (AS) components incorporated into the polymer matrix. For example, the AS can be diatomite, perlite, hydrated obsidian, diatomaceous earth, ash (e.g., volcanic ash or fly ash) or a mixture thereof. In certain aspects, the AS is a clay mineral phyllosilicate, such as alloysite, kaolinite, illite, montmorillonite, vermiculite, talc, palygorskite or pyrophyllite. An AS can also be a chlorite or a mica, such as biotite, muscovite, phlogopite, lepidolite, margariteor glauconite. AS components can be hydrated silicas. However, in certain cases, the AS is a expanded, such as by exposure of the silica in an oven.

The amount of AS used in the matrix can be varied depending on the application, but generally an aqueous slurry of at least 10% (by volume) AS will be used in preparing the matrix. For example, the slurry can be from about 30%-70% AS, such as about 40%, 50%, 60% AS or any intermediate percentage. The AS components for the slurry can comprise single type of AS or may be a mix two or more AS components. For example, a slurry could be used for matrix formation that comprises a equal mixture of perlite and vermiculite.

Surfactants

Surfactants, Surface-active materials, can, in some cases, be added to prepolymer compositions. Addition surfactants can be used to help control the size and shape of the foam cells by stabilizing the gas bubbles formed during nucleation. Surfactants can also aid in controlling the amount of cell opening and adjust shrinkage or reduced permeability.

A wide range of polymers may be used in a polymer matrix according to the invention. Suitable surfactants include anionic, cationic, dipolar-ionic (zwitterionic), ampholytic and nonionic surfactants and emulsifiers. For example, the surfactant can be a block copolymers of oxyethylene and oxypropylene or a silicone glycol copolymer liquid surfactant. Silicone-polyether liquid copolymer surfactants, for example, are known to produce foams with small, fine cells (see, e.g., U.S. Pat. No. 5,104,909). Certain of these silicone glycol copolymer liquid surfactants, when into hydrophilic foam-forming compositions, the result in foams having rapid wet out. Additional examples of suitable surfactants are described in published application 2004/0170670 which is hereby incorporated by reference.

Surfactants are not, however, required for hydrophilic polymers. For example, methods for making such polymers without addition of any surfactants are described in U.S. Pat. No. 5,296,518, incorporated herein by reference.

Additional Components

A matrix according to the invention may comprise one or more additional components. Such components can be deposited onto a matrix after polymerization or may be added to slurry prior to or during matrix polymerization. In particular, a matrix may comprise fertilizers and/or nutrients that support plant health. Such fertilizers and/or nutrient may, for example, be dissolved in an aqueous buffer or provided as pellets that form part of a slurry during matrix formation. For example, ammonium or nitrate salts can be incorporated as a nitrogen source for plants. Likewise, a suitable phosphorus source can be included. In some aspects, the pH of the matrix environment may be adjusted by adding an acid, a base or a pH buffering agent.

In still further aspects components can be added to alter the mechanical properties of a matrix material. For example, as described above, AS can be added to matrix. In certain other aspects, a natural or synthetic fibers can be added to provide additional structure to the matrix.

Still further components can be added to maintain the health of plants embedded in the matrix including antioxidants, pesticides, herbicide (i.e., to prevent undesired plant growth in the matrix), antibiotics, plant hormone and antifungal agents. For example, if rooted plants are desired in a matrix material, plant rooting hormones may be added to the matrix. Likewise, if contamination with microorganisms is a potential problem antimicrobial or antifungal compounds can be added to the matrix.

For example antifungal agent for use according to the invention include tebuconazole, simeconazole, fludioxonil, fluquinconazole, difenoconazole, 4,5-dimethyl-N-(2-propenyl)-2-(trimethylsilyl)-3-thiophenecarboxamide (silthiopham), hexaconazole, etaconazole, propiconazole, triticonazole, flutriafol, epoxiconazole, fenbuconazole, bromuconazole, penconazole, imazalil, tetraconazole, flusilazole, metconazole, diniconazole, myclobutanil, triadimenol, bitertanol, pyremethanil, cyprodinil, tridemorph, fenpropimorph, kresoxim-methyl, azoxystrobin, ZEN90160, fenpiclonil, benalaxyl, furalaxyl, metalaxyl, R-metalaxyl, orfurace, oxadixyl, carboxin, prochloraz, trifulmizole, pyrifenox, acibenzolar-5-methyl, chlorothalonil, cymoaxnil, dimethomorph, famoxadone, quinoxyfen, fenpropidine, spiroxamine, triazoxide, BAS50001F, hymexazole, pencycuron, fenamidone, guazatine, and cyproconazole.

Anti-microbials that may be used according to the invention include vanillin, thymol, eugenol, citral, carbacrol, biphenyl, phenyl hydroquinone, Na-o-phenylphenol, thiabendazole, K-sorbate, Na-benzoate, trihydroxybutylphenone, and propylparaben.

### II. Plants and Plant Parts

A wide range of plants can be maintained in the growth substrates according to the invention. As used herein the term “plant” refers to plant seeds, plant cuttings, seedlings and in vitro plant cultures as well as mature plants. For example, bedding plants, flowers, ornamentals, vegetables and other container stock can be provided in the substrates. Plants may be rooted in the matrix or may remain un-rooted. In certain aspects, the plants comprised in a matrix are callused.

Substrates can comprise vegetable crops or a living portions thereof such as artichokes, kohlrabi, arugula, leeks, asparagus, lentils, beans, lettuce, beets, bok choy, malanga, broccoli, melons (e.g., muskmelon, watermelon, crenshaw, honeydew, cantaloupe), brussels sprouts, cabbage, cardoni, carrots, napa, cauliflower, okra, onions, celery, parsley, chick peas, parsnips, chicory, peas, chinese cabbage, peppers, collards, potatoes, cucumber, pumpkins, cucurbits, radishes, dry bulb onions, rutabaga, eggplant, salsify, escarole, shallots, endive, soybean, garlic, spinach, green onions, squash, greens, sugar beets, sweet potatoes, turnip, swiss chard, horseradish, tomatoes, kale, turnips, and a variety of herbs.

Likewise, fruit and/or vine crops can be provided such as apples, apricots, cherries, nectarines, peaches, pears, plums, prunes, quince almonds, chestnuts, filberts, pecans, pistachios, walnuts, citrus, blackberries, blueberries, boysenberries, cranberries, currants, loganberries, raspberries, strawberries, grapes, avocados, bananas, kiwi, persimmons, pomegranate, pineapple, and other tropical fruits.

In certain preferred aspects, plant ornamental plants (or living portions thereof) are provided in substrate according to the inventions. For example, a matrix can comprise a plant such as an agastache, angelonia, antirrhinum, argyrantheum, bacopa, begonia, bidens, calibrachoa, coleus, crossandra, impatiens, diascia, fuchsia, gaura, gazania, geranium, helichrysum, ipomoea, kalanchoe, lamium, lantana, lavender, lobelia, nemesia, daisy, scaevola, oxalis, petunia, hibiscus, poinsettia, salvia, torenia, verbena, or viola plant. In still further aspects, the plant can be a cactus or other succulent.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Production of Synthetic Plant Growth Substrates

For formulations of plant growth substrates a slurry is initially mixed with polymer solution. A slurry of water and amorphous silica, such as expanded perlite and vermiculite, can formulated. Nutrients (e.g., nitrogen and phosphorus sources) and other additional components such as surfactants are added to the slurry as desired.

The slurry is then mixed with polyurethane prepolymer subunits and mechanical mixing is commenced to provide a homogenous slurry solution. Once homogenized the slurry solution is cast into molds and allowed to polymerize in open air.

Total polymer volume typically exceeds two-fold relative to the slurry volume. The resulting sponge-like polymer be arranged in trays. For example, strips of 10 or more individual plugs of polymer matrix can be arranged the tray. Optionally, the molded polymer and be further processed to the desired size of plug.

Plants or plant cuttings are embedded into the polymer matrix, such that moister and nutrients maintained in the sponge-like substrate are provide to the plant material. Plants can thus be maintained in the polymer matrix over extended time periods without desiccation.

### Example 2

### Physical Properties of Plant Media

The physical properties of three plant media, Rockwool® Propagation Cubes (“Rockwool”), a polymer matrix plug according to the embodiments (termed “Ecke”), and Oasis Wedge® Growing Medium (“Oasis”), were compared. The volume of Rockwool Propagation Cubes and the Ecke plugs were determined using cellophane/tape molds. The volume of Oasis plugs was determined by taping off the holes in the bottom of the Oasis trays and taping off small sections within the tray that are not occupied by the media. Total porosity was determined by placing the moist plugs in molds then slowly saturating the media.

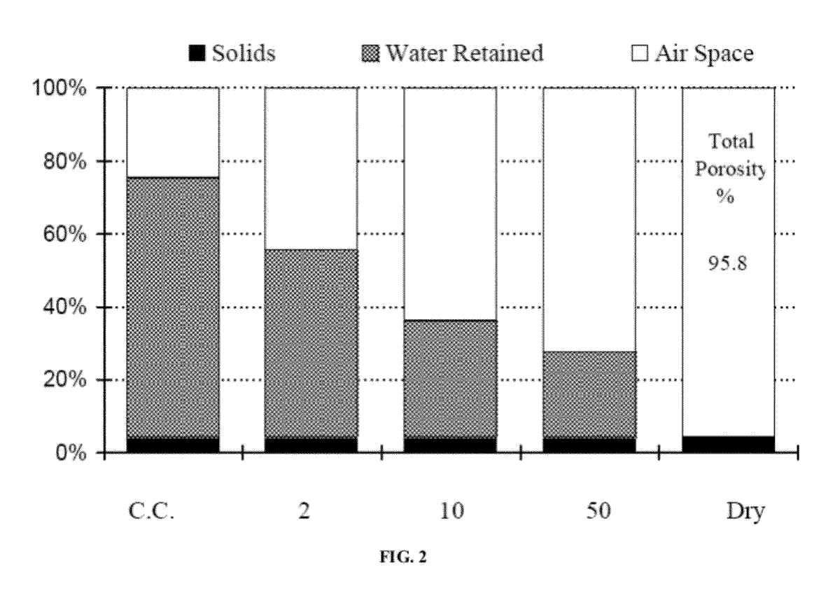

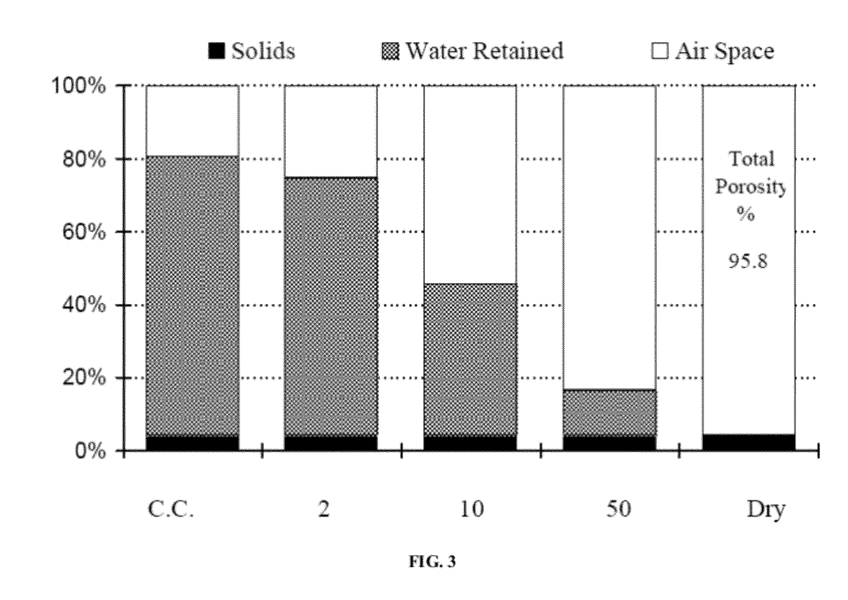

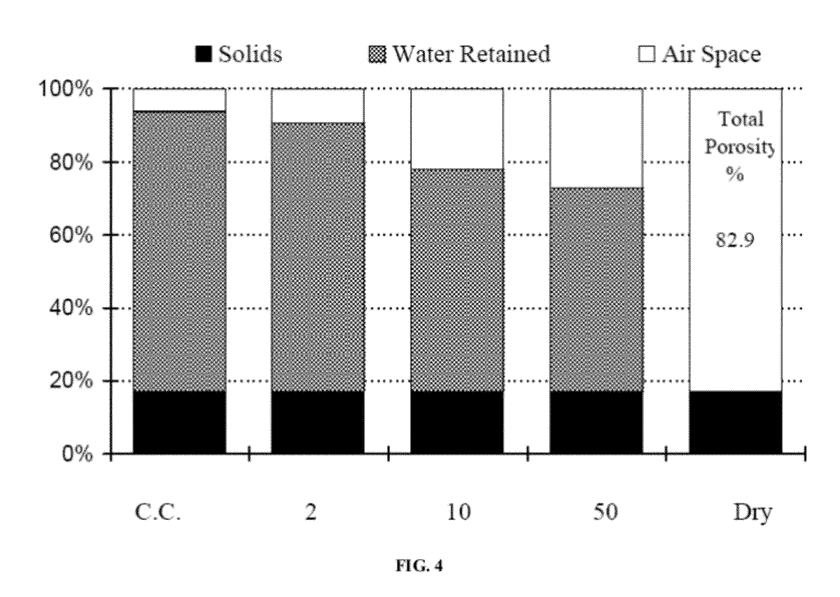

A summary of study results is presented in Table 1. Details of the physical properties obtained from each sample are presented in Tables 2-4 and the graphs of FIGS. 2-4. In each case the left most column is the container capacity column. After the media was saturated with water the column is pulled out of the water and the soil drains to the point of container capacity. The left most column of the bar graphs of FIGS. 2-4 represents the volume percentage of air, water and solid media after the free drainage.

Air space volume percentage at container capacity of at least 10% is usually desired; less air space can result in root suffocation and greater incidence of root rot. It is not uncommon, however, for successful seedling mixes tested in a shallow column to have less than 10% air space after drainage.

After free drainage the plug is subjected to pressure (2, 10 and 50 centibars) in a pressure plate apparatus with a ceramic plate. The 50 centibars tension represents an estimate of the limit at which plants can easily extract water. The difference between water held at 50 centibars and container capacity is expressed as readily available water.

TABLE 1
Summary of water retention results for tested media.

| | Ecke | Oasis | Rockwool |
|---|---|---|---|
| Volume of plug, cubic centimeter (CC) | 25 | 37 | 42 |
| CC of water held at container capacity | 18 | 29 | 32 |
| CC of was held at 2 centibars | 13 | 26 | 31 |
| CC water held at 10 centibars | 8 | 15 | 26 |
| CC water held at 50 centibars | 6 | 5 | 24 |
| CC of Readily Available Water | 12 | 24 | 9 |
| (Difference in water held at container | | | |
| capacity of 50 cb tension) | | | |

TABLE 2
Summary physical properties for Ecke media.

| Centibars suction | C.C.* | 2 | 10 | 50 | Dry |
|---|---|---|---|---|---|
| Density, lbs./cu.ft. | 53.1 | 40.4 | 28.3 | 23.0 | 8.4 |
| Water Retention, vol. % | 71.1 | 51.3 | 31.9 | 23.4 | 0.0 |
| Air Space, vol. % | 24.6 | 44.4 | 63.9 | 72.4 | 95.8 |
| Water Retention, % dry wt | 509 | | | | |

| | | |
|---|---|---|
| Readily available water, vol. % | 47.8 | Ecke Plug In |
| (Container capacity to 50 cb) | | Standard Tray |
| *Container capacity determined | | |
| using standard cube. | | |
| Potentially available water, vol. % | 59.4 | |
| (Readily available plus half that | | |
| held at 50 cb) | | |

| | |
|---|---|
| Saturated Bulk Density | 68.5 lbs./cu. ft. |

TABLE 3
Summary physical properties for Oasis media.

| Centibars suction | C.C.* | 2 | 10 | 50 | Dry |
|---|---|---|---|---|---|
| Density, lbs./cu.ft. | 48.9 | 45.1 | 26.9 | 8.8 | 1.1 |
| Water Retention, vol. % | 76.5 | 70.5 | 41.4 | 12.4 | 0.0 |
| Air Space, vol. % | 19.3 | 25.3 | 54.4 | 83.4 | 95.8 |
| Water Retention, % dry wt | 4228 | | | | |

| | | |
|---|---|---|
| Readily available water, vol. % | 64.1 | Standard Oasis |
| (Container capacity to 50 cb) | | Wedge Tray |
| *Container capacity determined | | |
| using standard cube. | | |
| Potentially available water, vol. % | 70.3 | |
| (Readily available plus half that | | |
| held at 50 cb) | | |

| | |
|---|---|
| Saturated Bulk Density | 60.9 lbs./cu. ft. |

TABLE 4
Summary physical properties for Rockwool media.

| Centibars suction | C.C.* | 2 | 10 | 50 | Dry |
|---|---|---|---|---|---|
| Density, lbs./cu.ft. | 51.7 | 50.8 | 42.9 | 39.7 | 5.0 |
| Water Retention, vol. % | 76.7 | 73.4 | 60.7 | 55.6 | 0.0 |
| Air Space, vol. % | 6.2 | 9.5 | 22.2 | 27.3 | 82.9 |
| Water Retention, % dry wt | 1235 | | | | |
| Readily available water, vol. % | 21.1 | | | | |
| (Container capacity to 50 cb) | | | | | |
| *Container capacity determined | | | | | |
| using standard cube | | | | | |
| Potentially available water, vol. % | 48.9 | | | | |
| (Readily available plus half that | | | | | |
| held at 50 cb) | | | | | |

| | |
|---|---|
| Saturated Bulk Density | 55.6 lbs./cu. ft. |

Thus, results indicate that all three media wetted up easily. The Ecke and Oasis had similar drainage and water holding characteristics in this study. Both had a high degree of airspace after free drainage and both released most of the water at tensions below 50 cb. The Rockwool plug has a slightly lower total porosity and less airspace after free drainage. The Rockwool held water more tightly with the majority of the moisture in the cube retained at 50 cb of tension. A portion of the water held at 50 cb will be available to the plant but the extraction of the water may place the plants under osmotic stress.

### REFERENCES

The following references, to the extent that they provide exemplary procedural or other details supplementary to those set forth herein, are specifically incorporated herein by reference.
U.S. Pat. No. 3,903,232
U.S. Pat. No. 4,137,200
U.S. Pat. No. 4,517,326
U.S. Pat. No. 5,650,450
U.S. Pat. No. 5,916,928
U.S. Pat. No. 6,032,412
U.S. Pat. No. 6,901,699
U.S. Pat. No. 7,832,145
U.S. Patent Publn. 2004/0020110
U.S. Patent Publn. 2008/0035217
U.S. Patent Publn. 2012/0005955
Dutch Patent Appln. NL-2004951

1. A sponge-like matrix that is porous, retains water and can be used to maintain plant growth, the matrix comprising an admixture of a hydrophilic polymer and an amorphous silica, wherein the matrix is essentially free of organic soil, peat or bark material.

2. The matrix of claim 1, wherein the matrix comprises an average porosity of between about 10 and 300 pores per inch (ppi).

3. The matrix of claim 1, wherein the hydrophilic polymer is a polyurethane polymer.

4. The matrix of claim 3, wherein the polyurethane polymer is a polymer of a polyol and an isocyanate.

5. The matrix of claim 4, wherein the polyurethane polymer has a molecular weight (MW) between branch points of between about 1,000 and 10,000.

6. The matrix of claim 4, wherein the polymer is a polymer of isocyanate and further wherein the isocyanate is toluene diisocyanate (TDI) methylene diphenyl diisocyanate (MDI), hexamethylene diisocyanate (HDI) or isophorone diisocyanate (IPDI).

7. The matrix of claim 6, wherein the TDI is a mixture of 2,4-TDI and 2,6-TDI.

8. The matrix of claim 6, wherein the MDI is 4,4′-MDI.

9. The matrix of claim 6, wherein the MDI is polymeric MDI.

10. The matrix of claim 1, wherein the polymer is a polymer of a polymeric polyol.

11. The method of claim 10, wherein the polymeric polyol is a polyether polyol or polyester polyol.

12. The matrix of claim 1, wherein the amorphous silica is vermiculite, biotite, phlogopite, mica, perlite, hydrated obsidian or diatomaceous earth.

13. The matrix of claim 1, wherein the amorphous silica comprises amorphous silica particles dispersed in the polymer.

14. The matrix of claim 1, wherein the amorphous silica is a hydrated silica.

15. The method of claim 1, wherein the amorphous silica is expanded vermiculite or expanded perlite.

16. The matrix of claim 1, wherein the matrix is shaped in the form of a plug, pot or slab.

17. The matrix of claim 16, wherein the matrix is shaped in the form of plug and the plug comprises (a) a centrally disposed cavity or (b) a cut that bisects all or a portion of the plug.

18. The matrix of claim 1, comprising a nitrogen source, a phosphorus source, a surfactant, a pesticide, an herbicide, an antibiotic or an antifungal agent.

19. The matrix of claim 18, wherein the nitrogen source is an ammonium or nitrate salt.

20. The matrix of claim 1, comprising a plant hormone.

21. The matrix of claim 1, comprising a plant.

22. The method of claim 21, wherein the plant is a plant seed, a plant seedling, an in vitro plant culture or a cutting from a plant.

23. The method of claim 21, wherein the plant is a monocot.

24. The matrix of claim 21, wherein the plant is a dicot.

25. The matrix of claim 21, wherein the plant is an ornamental plant, a landscaping plant, an herb, a garden vegetable or a fruit or nut tree.

26. The matrix of claim 25, wherein the plant is a poinsettia, impatiens or geranium plant.

27. A method for growing a plant comprising positioning a plant in a matrix according to claim 1 and allowing the plant to grow.

28-35. (canceled)

36. A method for transplanting a plant comprising:
(a) obtaining a first container comprising a plant positioned in a matrix according to claim 1; and
(b) transplanting the plant and matrix to a second container.

37-39. (canceled)
